(19) United States
(12) Patent Application Publication
(10) Pub. No.: US 2013/0331728 A1
(43) Pub. Date: Dec. 12, 2013

(54) METHOD AND APPARATUS FOR DETERMINING A CORE TEMPERATURE OF AN INTERNAL ORGAN

(71) Applicants: Hoi-Cheong S. Sun, Tampa, FL (US); Wajeeh M. Saadi, Tampa, FL (US)

(72) Inventors: Hoi-Cheong S. Sun, Tampa, FL (US); Wajeeh M. Saadi, Tampa, FL (US)

(73) Assignee: The Charles Stark Draper Laboratory, Inc.

(21) Appl. No.: 13/490,176

(22) Filed: Jun. 6, 2012

(51) Int. Cl. A61B 5/01

(52) U.S. Cl. 600/549

(57) ABSTRACT

Systems and methods of determining a core temperature of an internal organ in a body are provided. A thermal mass of known thermal resistance can be located between a body-contacting substrate and an external substrate. The body-contacting portion of the substrate can be placed over the organ whose temperature is to be measured. Temperature sensors can measure a temperature difference across the thermal mass and an absolute temperature at the external portion of the substrate. An electronic processor can receive the temperature measurements and can compute the core temperature of the organ based on the temperature measurements and the thermal resistances of the thermal mass and the tissue surrounding the organ.

### BACKGROUND

Core temperature is the operating temperature of an internal organ in the body, such as the heart or liver. In humans, the core temperature must remain within a narrow range in order to maintain organ health. Patients whose core temperatures deviate from the normal range can be at risk of permanent organ damage or death. Typically, an invasive procedure such as insertion of a temperature probe directly into the organ of interest is used to determine the core temperature. A less invasive procedure involves the patient ingesting a pill that measures the temperature as it travels through the digestive tract, but this method sacrifices the ability to control the location of the temperature sensor over a long period of time. It is desirable for physicians and patients to have accurate, noninvasive methods for determining the core temperature of internal organs.

### SUMMARY OF THE INVENTION

Aspects and implementations of the present disclosure are directed to systems and methods of determining a core temperature of an internal organ in a body.

At least one aspect is directed to an apparatus for measuring a core temperature of an internal organ in a body. The apparatus includes a substrate material having a body-contacting portion positioned over the organ of interest and an external portion opposed to the body-contacting portion and not in contact with the body. The apparatus includes a thermal mass of known thermal resistance, located between the body-contacting and external portions of the substrate. The apparatus includes a differential temperature sensor for measuring a temperature difference between the body-contacting portion and the external portion of the substrate, and an absolute temperature sensor for measuring the temperature at the external portion of the substrate. The apparatus also includes an electronic processor configured to receive the temperature information from the temperature sensors and calculate the core temperature based on the differential and absolute temperatures and the thermal resistances of the thermal mass and tissue surrounding the organ.

At least one aspect is directed to a method for determining a core temperature of an internal organ in a body. The method includes the step of measuring, by a differential temperature sensor, a temperature difference across a thermal mass of known thermal resistance surrounded by a substrate having a first portion in contact with the body and a second portion not in contact with the body. The method includes the step of measuring, by an absolute temperature sensor, an absolute temperature at the external portion of the substrate. The method includes the step of receiving, by an electronic processor, information corresponding to the temperatures measured by the differential and absolute temperature sensors. The method also includes the step of calculating, by the electronic processor, a core temperature of the internal organ based on the temperatures measured by the differential and absolute temperature sensors and the thermal resistances of the thermal mass and the tissue surrounding the organ.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a depiction of an illustrative implementation of a core temperature sensor for determining core temperature of an internal organ, shown measuring the temperature of a patient's liver;

FIG. 2A is a cross-sectional view of a core temperature sensor for determining the core temperature of an internal organ, according to an illustrative implementation;

FIG. 2B is a top view of the apparatus shown in FIG. 2A;

FIG. 2C is a bottom view of the apparatus shown in FIG. 2A;

FIG. 3A is a cross-sectional view of a second core temperature sensor for determining the core temperature of an internal organ, according to another illustrative implementation;

FIG. 3B is top view of the apparatus shown in FIG. 3A;

FIG. 3C is a bottom view of the apparatus shown in FIG. 3A;

FIG. 4 is a circuit model of a core temperature sensor for determining a core temperature of an internal organ as depicted in FIGS. 2A-C, according to an illustrative implementation;

FIG. 5 is a circuit model of a core temperature sensor for determining a core temperature of an internal organ as depicted in FIGS. 3A-C, according to an illustrative implementation;

FIG. 6 is a flow chart of a method of determining a core temperature of an internal organ, according to an illustrative implementation.

### DESCRIPTION OF CERTAIN ILLUSTRATIVE IMPLEMENTATIONS

Following below are more detailed descriptions of various concepts related to, and implementations of, methods and systems for determining a core temperature of an internal organ in a body. The various concepts introduced above and discussed in greater detail below may be implemented in any of numerous ways, as the described concepts are not limited to any particular manner of implementation. Examples of specific implementations and applications are provided primarily for illustrative purposes.

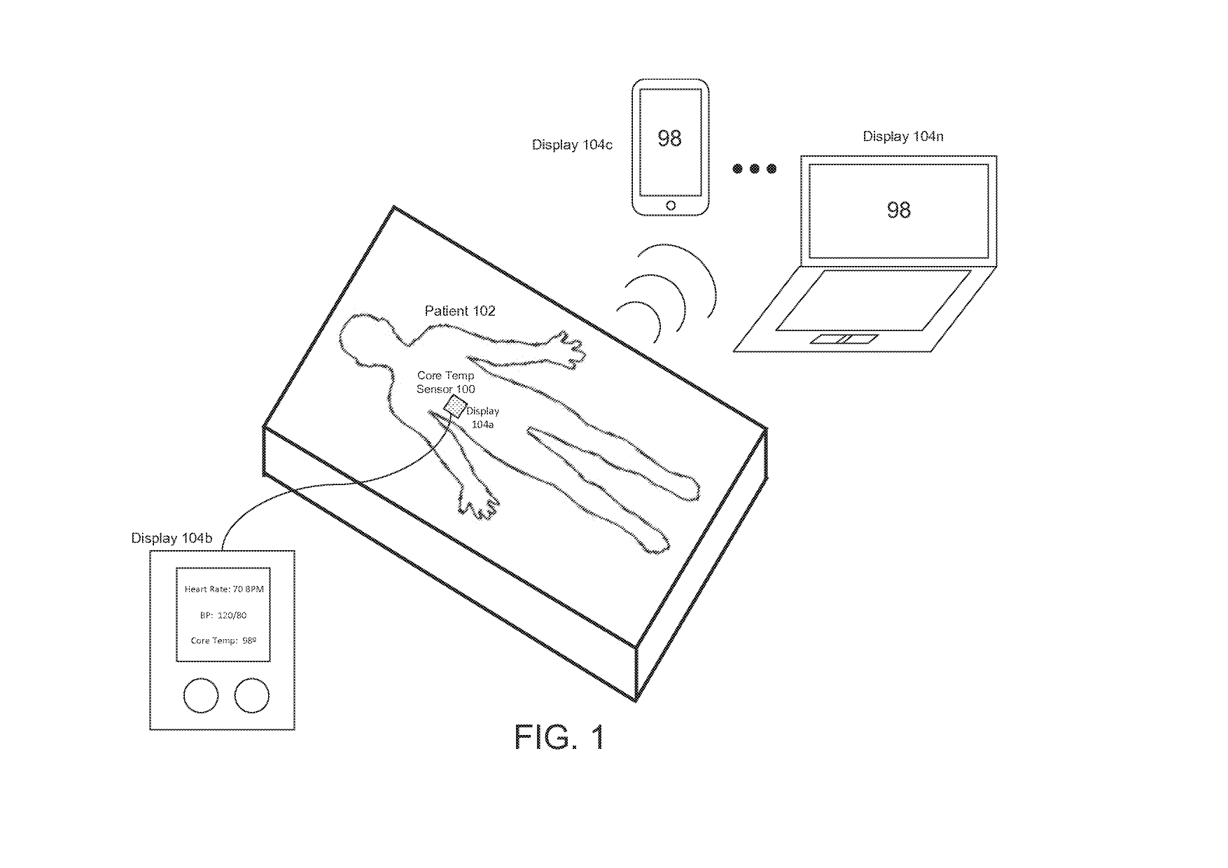

FIG. 1 is a schematic diagram of a core temperature sensor 100 for measuring a core temperature of an internal organ, depicted in use in a hospital setting. The core temperature sensor 100 is placed in contact with the skin of the patient 102, and positioned over the organ whose temperature is to be measured. As shown in the figure, the core temperature sensor 100 is relatively small and is able to determine the core temperature of an organ without the need for an invasive medical procedure. The core temperature sensor 100 is removably attached to the skin of the patient 102 using an adhesive.

The core temperature sensor 100 communicates with any number of the display devices 104a-104n. Communication between the core temperature sensor 100 and the display devices 104a-104n can be achieved using any wired or wireless communication protocol. The display devices 104a-104n can be any device suitable for displaying temperature information. For example, the display device 104 can be built into the core temperature sensor 100 (e.g. display 104a). The display device 104 can also be a medical monitor 104b, a handheld electronic device 104c, or a desktop or laptop computer 104n.

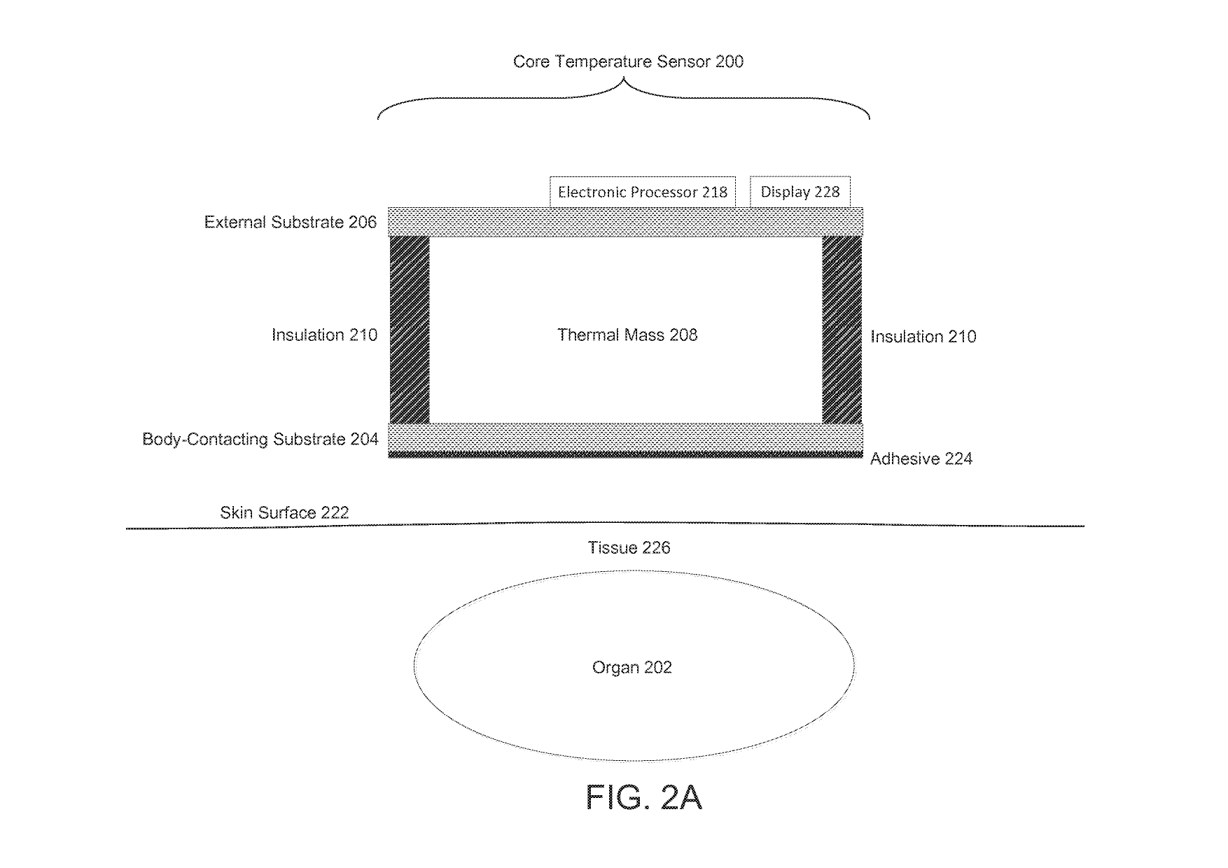

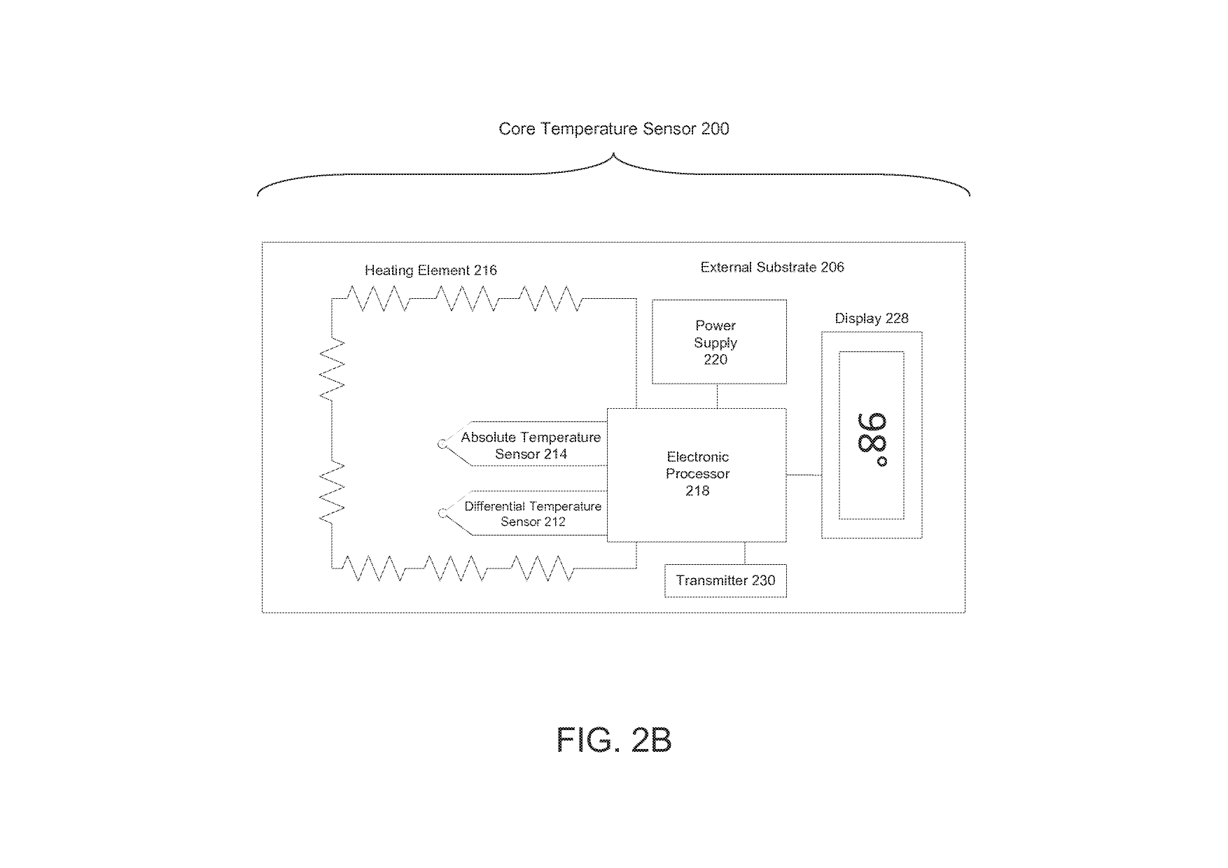

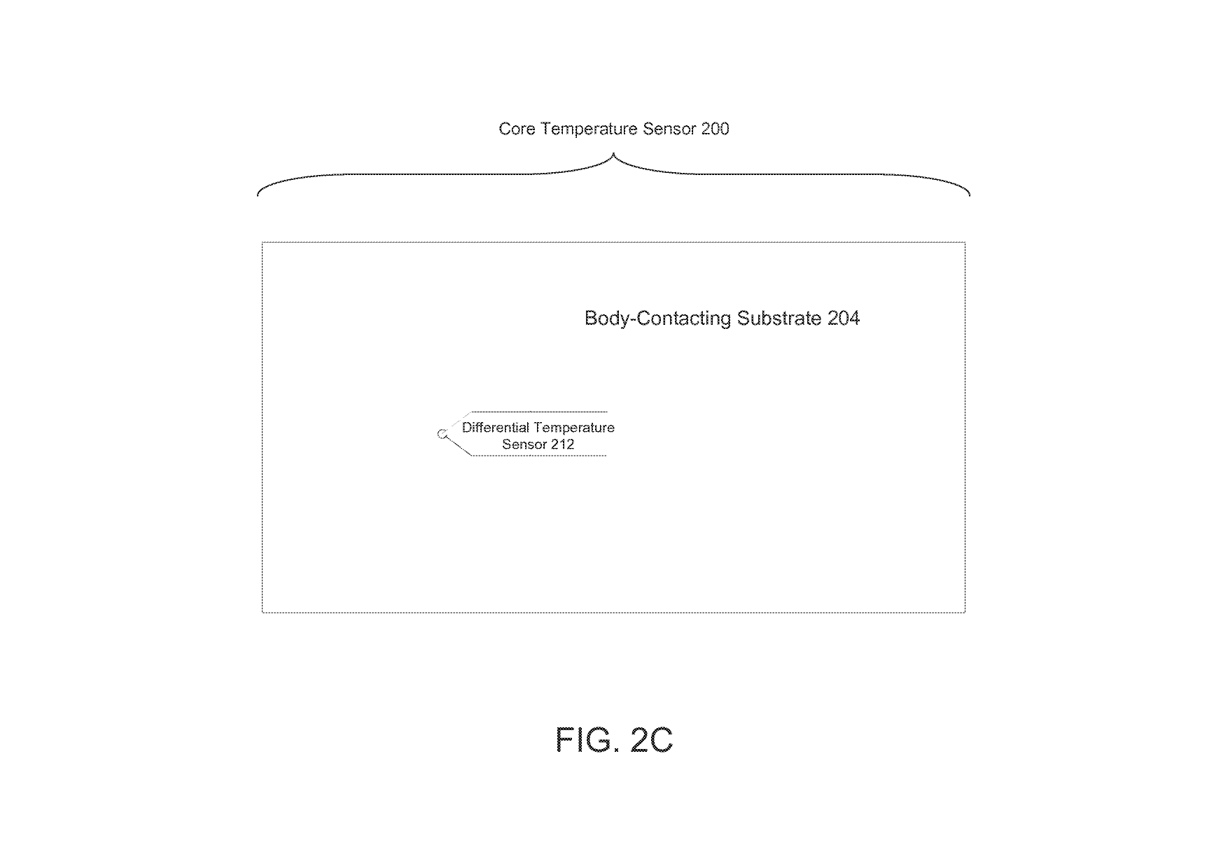

FIGS. 2A-C depict various views of a core temperature sensor 200 suitable for use as the core temperature sensor 100 of FIG. 1. FIG. 2A shows a cross-sectional view, FIG. 2B shows a top view, and FIG. 2C shows a bottom view of the core temperature sensor 200. The core temperature sensor 200 includes a thermal mass 208, a differential temperature sensor 212, an absolute temperature sensor 214, and a heating element 216 mounted to a pair of substrates (a body-contacting substrate 204 and an external substrate 206).

The thermal mass 208 is located between the body-contacting substrate 204 and the external substrate 206, and is composed of a heat conducting material whose thermal resistance is known. Heat generated by the organ 202 will dissipate through the thermal mass 208 after traveling first through the tissue 226 and the skin surface 222. If the temperature at the skin surface 222 differs from the temperature of the surrounding environment, there will be a non-zero temperature difference across the thermal mass 208. The material of the thermal mass can be selected to substantially match the thermal resistance of the tissue 226 surrounding the organ 202. For example, the material can be chosen based on the particular kind of organ whose temperature is to be measured (e.g. heart, liver, etc.) or the body mass index of the patient. In some implementations, the thermal mass 208 is a plastic material such as silicone, polycarbonate, polystyrene, polypropylene, polyamide, polyimide, polyethylene, polysulfone, polytetrafluoroethylene, and the like. In other implementations, the thermal mass 208 can be composed of a material whose thermal mass is variable. For example, the core temperature sensor 200 can include a microfluidic pump coupled to a reservoir containing liquid whose thermal resistance is different from the thermal resistance of the thermal mass 208. The liquid can be transported into the thermal mass 208 by the microfluidic pump. Thus, the overall thermal resistance of the thermal mass 208 can vary in proportion to the amount of liquid pumped into the thermal mass 208. In one implementation, the thermal mass 208 is composed of a porous material capable of absorbing liquid pumped into the thermal mass 208 by the microfluidic pump.

A thermal insulation material 210 surrounds the exposed sides of the thermal mass 208. The insulation 210 thermally isolates the exposed surface of the thermal mass from the environment so that the heat conducted through the thermal mass 208 is generated primarily by the organ 202. The insulation 210 adjoins the body-contacting substrate 204 and external substrate 206, so that the thermal mass 208 is completely enclosed and not exposed to the surrounding environment other than through the body-contacting substrate 204 and the external substrate 206.

The differential temperature sensor 212 detects the total temperature difference across the thermal mass 208. In one implementation, the differential temperature sensor is a thermopile whose ends are located in the body-contacting substrate 204 and the external substrate 206, at either side of the thermal mass 208. For example, the probe shown in FIG. 2C is located on the bottom surface of the core temperature sensor 200, coupled to the body-contacting substrate 204 to be placed near or on the surface of the body. The probe shown in FIG. 2B is on the top surface of the core temperature sensor 200, coupled to the external substrate 206 and away from the surface of the body.

The absolute temperature sensor 214 measures an absolute temperature at an external portion of the core temperature sensor 200. For example, in one implementation, the absolute temperature sensor 214 is a thermistor coupled to the external substrate 206 on the top surface of the core temperature sensor 200, as shown in FIG. 2B. The temperature measured by the absolute temperature sensor 214 is based in part on the heat emanating from the organ 202 through the tissue 226 and the thermal mass 208, and in part on the temperature of the environment surrounding the core temperature sensor 200. This temperature is also impacted by the heating element 216.

The heating element 216 is configured to apply a known reference heat to an external portion of the core temperature sensor 200. FIG. 2B shows the heating element 216 as a resistive trace embedded into or deposited on the external substrate 206 on the top surface of the core temperature sensor 200. This design allows the heat applied by the heating element 216 to be evenly distributed on the top surface of the core temperature sensor 200. The amount of heat applied is proportional to the current travelling through the heating element 216. In some implementations, the reference heat is a constant temperature. In other implementations, the reference heat changes over time, for example, according to a preselected modulation scheme.

The core temperature sensor 200 also includes an electronic processor 218, a power supply 220, and a display device 228, as shown mounted to the external substrate 206 in FIGS. 2A-B. The electronic processor 218 is communicatively coupled to the differential temperature sensor 212, the absolute temperature sensor 214, the heating element 216, and the display 228. The electronic processor 218 receives information corresponding to the temperature difference measured by the differential temperature sensor 212 and the absolute temperature measured by the absolute temperature sensor 214. Instructions relating to the magnitude and duration of the reference heat to be applied to the external portion of the core temperature sensor 200 are transmitted by the electronic processor 218 to the heating element 216 or a driver coupled thereto. Based on the known thermal resistances of the thermal mass 208 and the tissue 226 surrounding the organ 202, as well as the received temperatures measured by the differential temperature sensor 212 and the absolute temperature sensor 214, the electronic processor 218 calculates the core temperature of the organ 202, as described further below. The electronic processor receives electrical power from the power supply 220. The power supply 220 also supplies electrical power to the heating element 216 and the display device 228.

The electronic processor 220 transmits information corresponding to the calculated core temperature to the display device 228. As shown in FIG. 2B, the electronic processor can communicate with a display device 228 integrated into the core temperature sensor 200. In this example, the display device 228 is a display device that is suitable for use as display device 104a of FIG. 1. In other implementations, the electronic processor 218 is configured to transmit the core temperature to a remote display device, such as the medical monitor 104b, the handheld electronic device 104c, or the personal computer 104n of FIG. 1. The electronic processor 218 can communicate with display devices 104a-104n via any wired or wireless communication protocol via a transmitter 230 coupled to the electronic processor 218.

The display device 228 receives information corresponding to the calculated core temperature of the internal organ 202, and presents this information in textual or graphical form. The display 104 can provide temperature information to be used by a physician providing care to the patient 102 of FIG. 1, or by the patient 102 or other non medical professional. For example, the display 104c can be a mobile device allowing the patient 102 to monitor a core temperature in a home setting.

As mentioned above, the thermal mass 208, the differential temperature sensor 212, the absolute temperature sensor 214, the heating element 216, the electronic processor 220, the power supply 218, the display 228, and the transmitter 230 are mounted to either a body-contacting substrate 204 or an external substrate 206. Specifically, the core temperature sensor 200 includes a body-contacting substrate 204, an external substrate 206, and an adhesive 224. The body-contacting substrate 204 has a bottom surface for positioning on the skin 222 on the outside of the body, over an organ of interest 202. FIG. 2C is a view of the bottom surface of the core temperature sensor 200, showing the body-contacting substrate 204 and one probe of the differential temperature sensor 212. The top surface of the body-contacting substrate 204 is in contact with the thermal mass 208 as shown in FIG. 2A. In some implementations, the body-contacting substrate 204 is a thin material whose thermal resistance is negligible relative to the thermal resistance of the thermal mass 208. For example, the body-contacting substrate 204 can be composed of KAPTON, Teflon, polyester (PET) or polyethylene napthalate (PEN). An adhesive 224 can be applied to the bottom surface of the body-contacting substrate 204. The adhesive is a standard medical grade adhesive for removably attaching the core temperature sensor 200 to the skin surface 222.

The external substrate 206 is opposed to the body-contacting substrate 204, away from the skin surface 222. A bottom surface of the external substrate 206 is in contact with the thermal mass 208, so that the thermal mass 208 is enclosed on opposite ends by the body-contacting substrate 204 and the external substrate 206, as shown in FIG. 2A. The electronic processor 218 and a display 228 are attached to a top surface of the external substrate 206. Also included on the top surface of the external substrate 206 are the second probe of the differential temperature sensor 212, the absolute temperature sensor 214, the heating element 216, and the power supply 220, as shown in FIG. 2B. In some implementations, the material used to create the external substrate 206 can be the same as the material of the body-contacting substrate 204.

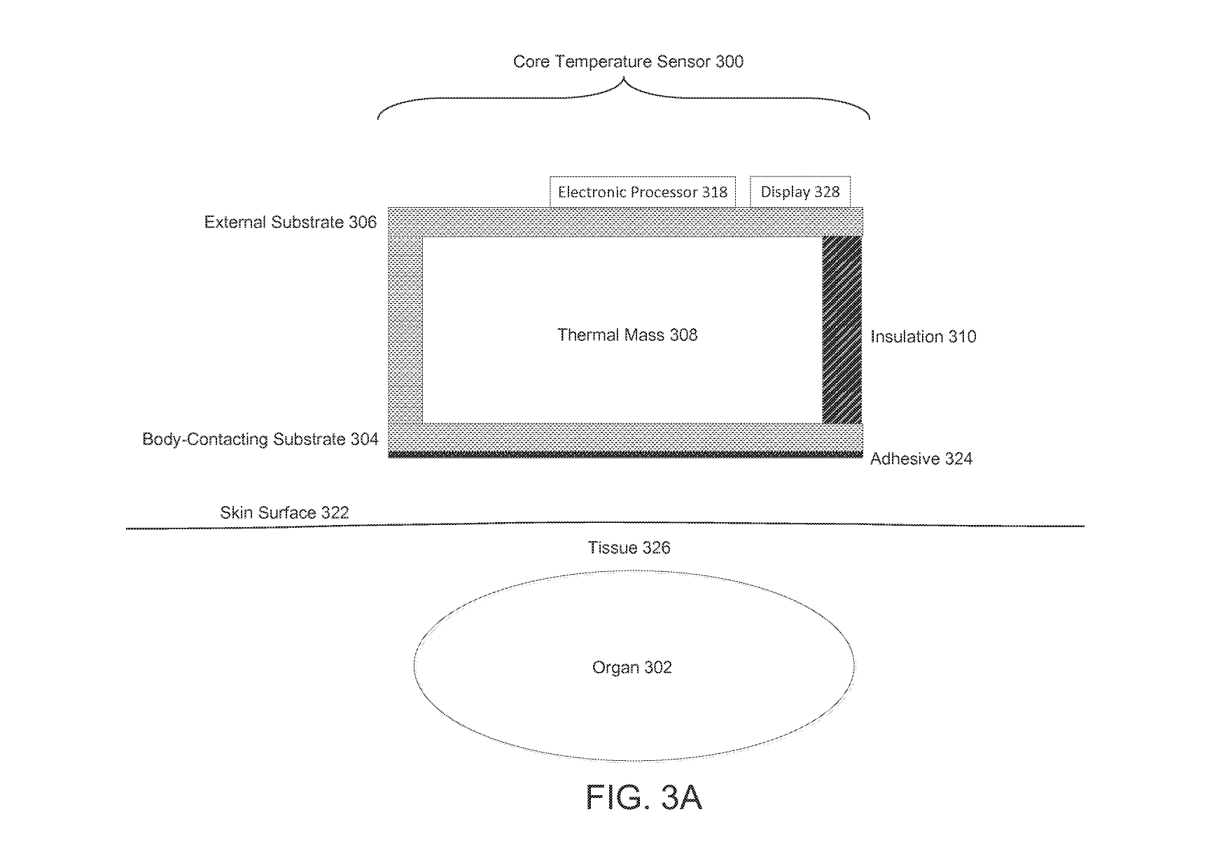

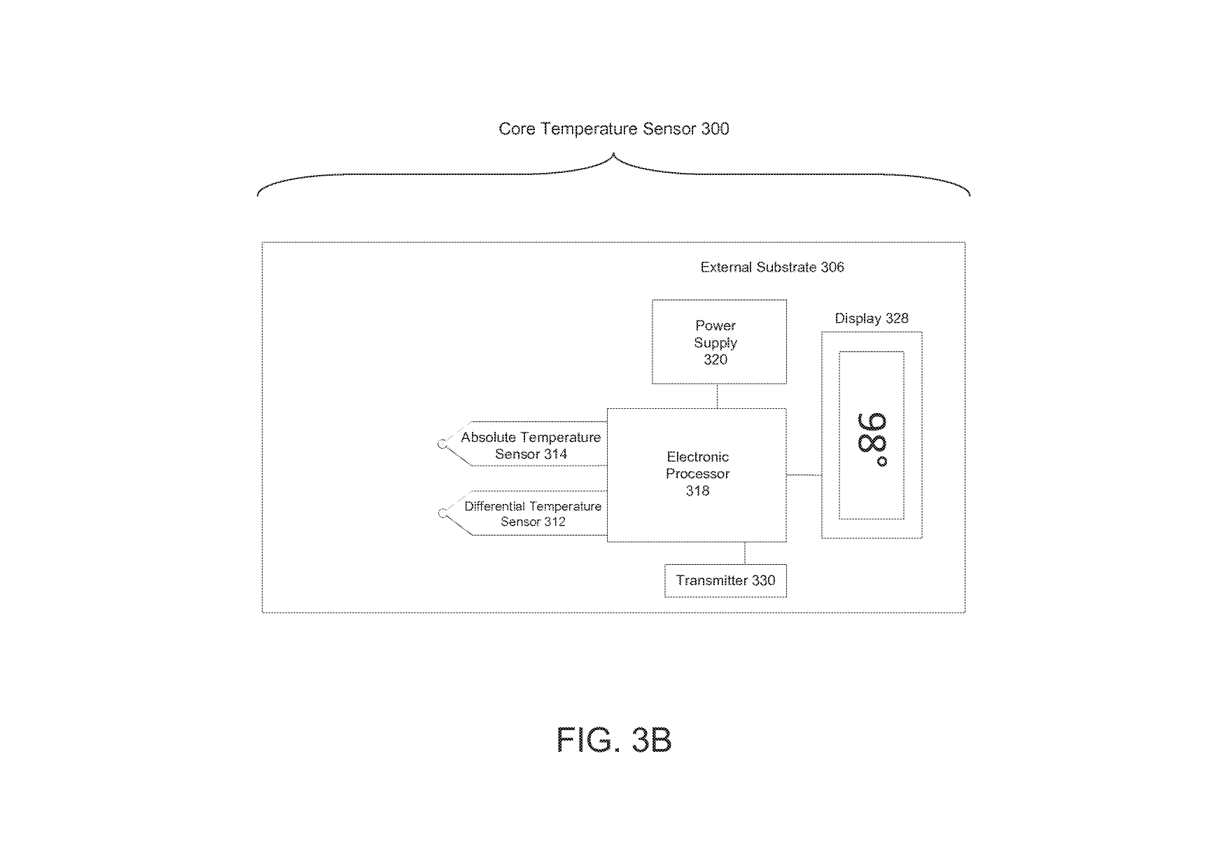

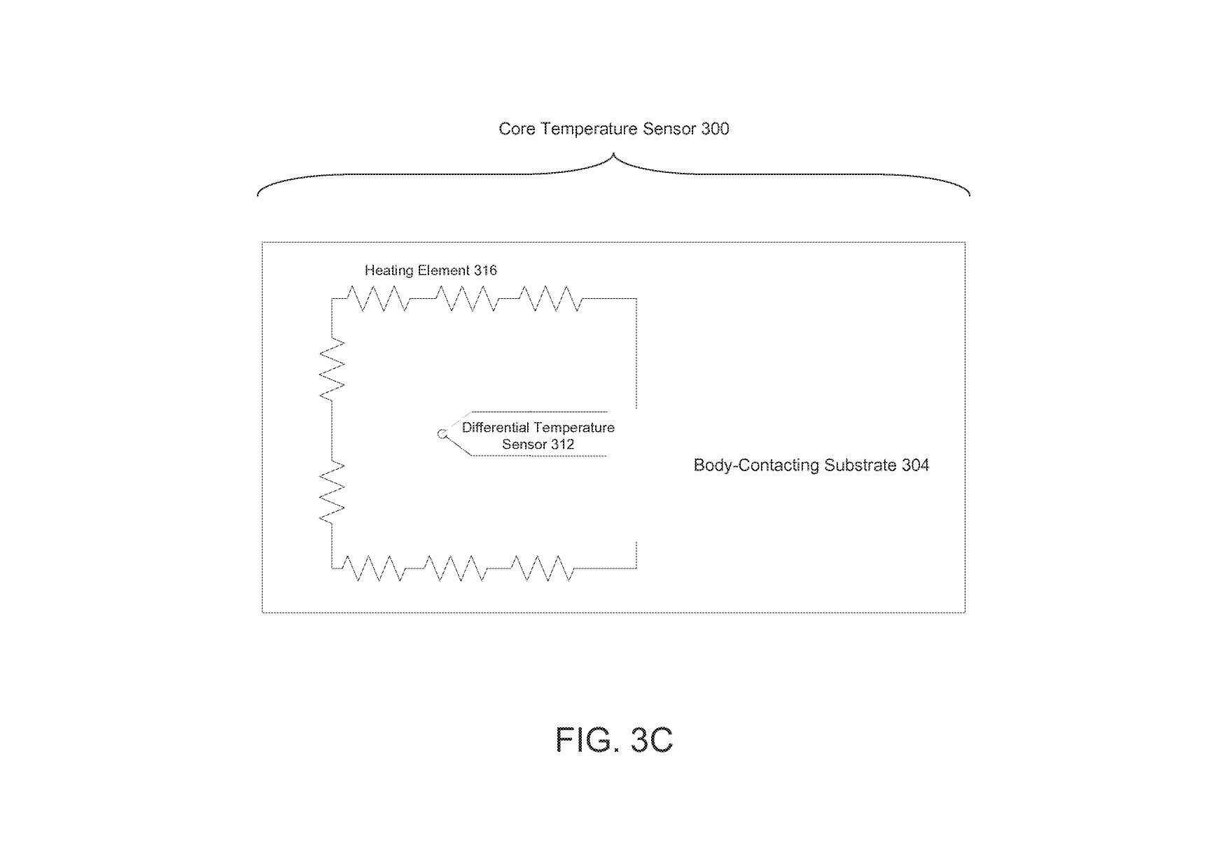

FIGS. 3A-C depict various views of a second core temperature sensor 300 suitable for use as the core temperature sensor 100 of FIG. 1. FIG. 3A shows a cross-sectional view, FIG. 3B shows a top view, and FIG. 3C shows a bottom view of the core temperature sensor 300. The elements of core temperature sensor 300 are substantially analogous to the elements of core temperature sensor 200, as described above in connection with FIGS. 2A-C. However, the core temperature sensor 300 uses an alternative substrate construction. In addition, the heating element 316, analogous to heating element 216, is formed on the body-contacting substrate 304 (analogous to the body-contacting substrate 204) instead of the external substrate 306 (analogous to the external substrate 206).

As shown in FIG. 3A, core temperature sensor 300 includes a body-contacting substrate 304 and an external substrate 306. The body-contacting substrate 304 is configured to be removable attached to the skin surface 322 via the adhesive 324. The external substrate 306 contains electronic components such as an electronic processor 318, a display 328, a power supply 320, a transmitter 330, a differential temperature sensor 312, and a absolute temperature sensor 314, as illustrated by FIGS. 3A-B, which are analogous to the electronic processor 218, the display 228, the power supply 220, transmitter 230, the differential temperature sensor 212, and the absolute temperature sensor 214, respectively. However, as shown in FIG. 3A, the body-contacting substrate 304 and the external substrate 306 are merely two portions of a single continuous piece of substrate material, which can facilitate electrical connection between components on the body-contacting substrate 304 and the external substrate 306.

The second difference between the core temperature sensor 200 of FIGS. 2A-C and the core temperature sensor 300 of FIGS. 3A-C is the location of the heating element 316. In FIGS. 2A-C, the heating element 216 is located on the top surface of the external substrate 306. As shown in FIGS. 3B-C, the heating element 316 is absent from the external substrate 306, and is instead located on the body-contacting substrate 304. The structure and function of the heating element 316 are the same as those of heating element 216. For example, the heating element 316 can be a resistive heating element used to apply a known, evenly distributed reference heat to the surface of the body-contacting substrate 304. The reference heat can be a constant heat level, or it can be a modulated heat signal. In some implementations, the reference heat is applied when the core temperature sensor 300 is first placed in contact with the skin surface 322, in order to determine the relative thermal resistances of the thermal mass 308 and the tissue 326. Subsequent use of the core temperature sensor 300 may not require further application of a reference heat by the heating element 316.

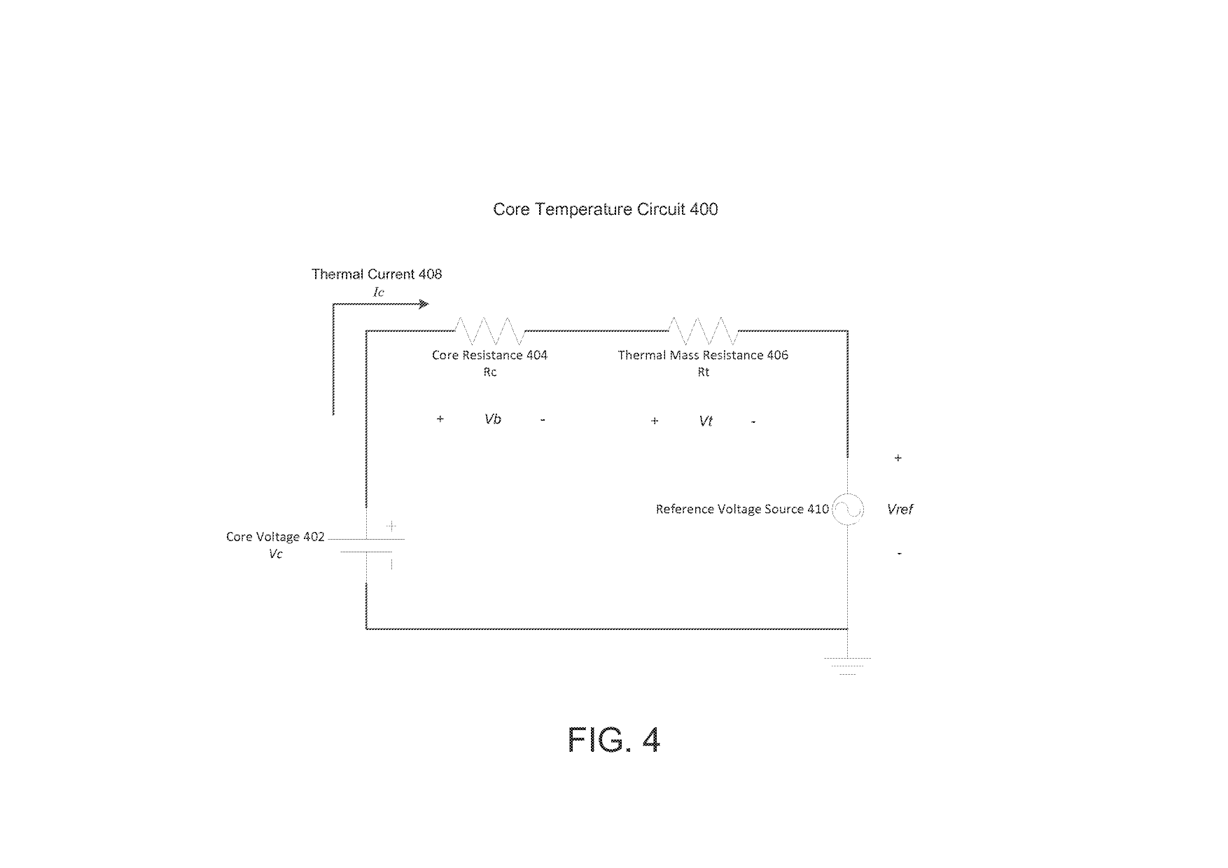

FIG. 4 is a schematic diagram of an electrical circuit 400 that is useful for modeling the behavior of the core temperature sensor 200 of FIGS. 2A-C, as it is used to determine a core temperature of the organ 202. The core temperature circuit 400 models temperatures as voltages, thermal resistances as electrical resistances, and thermal currents as electrical currents. The organ 202 generates heat, and is therefore modeled as a core voltage source 402 in FIG. 4. The tissue 226 surrounding the organ 202 conducts and dissipates heat, so the tissue 226 is modeled as core resistance 404. Similarly, the thermal mass 208 is represented by thermal mass resistance 406. Finally, the heating element 216 corresponds to reference voltage 410.

The arrangement of the elements in core temperature circuit 400 is analogous to the arrangement of the corresponding items shown in FIGS. 2A-C. For example, when the core temperature sensor 200 is placed in contact with the skin surface 222, alignment and sequential order are maintained between the organ 202, the tissue 226, and the thermal mass 208. The alignment and order are represented in the core temperature circuit 400 by electrically connecting the core voltage 402, the body resistance 404, and the thermal mass resistance 406 in series. The heating element 216 is located on the top surface of the thermal mass 208, and that arrangement is preserved in the core temperature circuit 400 as well. The thermal current 408 corresponds to the flow of heat generated by the organ 202 (core voltage 402), which moves first through the tissue 226 (core resistance 404) and then through the thermal mass 208 (thermal mass resistance 406). The voltage Vt in core temperature circuit 400 corresponds to the temperature readout from the differential temperature sensor 212, and the voltage Vref corresponds to the readout from the absolute temperature sensor 214.

Applying known circuit analysis techniques, an equation for the core temperature Vc can be derived as follows: Vc=IcRc+IcRt+Vref. According to Ohm's Law, the current k is equal to the voltage Vt divided by the thermal mass resistance Rt. Making this substitution, the equation for Vc becomes:

$$Vc = \frac{Vt}{Rt} Rc + Vt + Vref .$$

This equation can be simplified to yield the following equation:

$$Vc = Vt ( 1 + \frac{Rc}{Rt} ) + Vref .$$

Over a short duration of time, the temperature change of organ 202 in response to a temperature change in the surrounding environment is negligible. Thus, the heating element 216 can be controlled to apply a time-varying reference heat without significantly impacting the temperature of the organ 202. The following equation derived from core temperature circuit 400 describes the relationship between the reference heat applied by heating element 216 and the other circuit elements, including the temperature change to the organ 202 (core voltage 402):

$$Δ Vc = 0 = Δ Vt ( 1 + \frac{Rc}{Rt} ) + Δ Vref . Thus , - Δ Vref = ( 1 + \frac{Rc}{Rt} ) Δ Vt .$$

Finally, dividing both sides of the equation by ΔVₜ, the equation can be written as:

$$\frac{- Δ Vref}{Δ Vt} = ( 1 + \frac{Rc}{Rt} ) .$$

In the equation above, the value

$$\frac{- Δ Vref}{Δ Vt}$$

is the measurable response factor from the changing reference voltage Vref Substituting this value into the equations for Vc results in the following equation:

$$Vc = Vt ( \frac{- Δ Vref}{Δ Vt} ) + Vref .$$

The voltage levels in core temperature circuit 400 represent temperatures in the actual system. Temperature variables Tc, T1, and T2 can be substituted into the equation for Vc, where Tc corresponds to the core temperature of the organ 202, T1 corresponds to the reading on the absolute temperature sensor 214, and T2 corresponds to the reading on the differential temperature sensor 212. The substitution leads to the following equation, which can be used to determine the core temperature of the organ 202:

$$Tc = T 2 ( \frac{- Δ Vref}{Δ Vt} ) + T 1.$$

Because the reference voltage source 410 corresponds to the heating element 216, the value

$$\frac{- Δ Vref}{Δ Vt}$$

corresponds to the response factor from the reference heat applied by heating element 216. This response factor can be determined empirically.

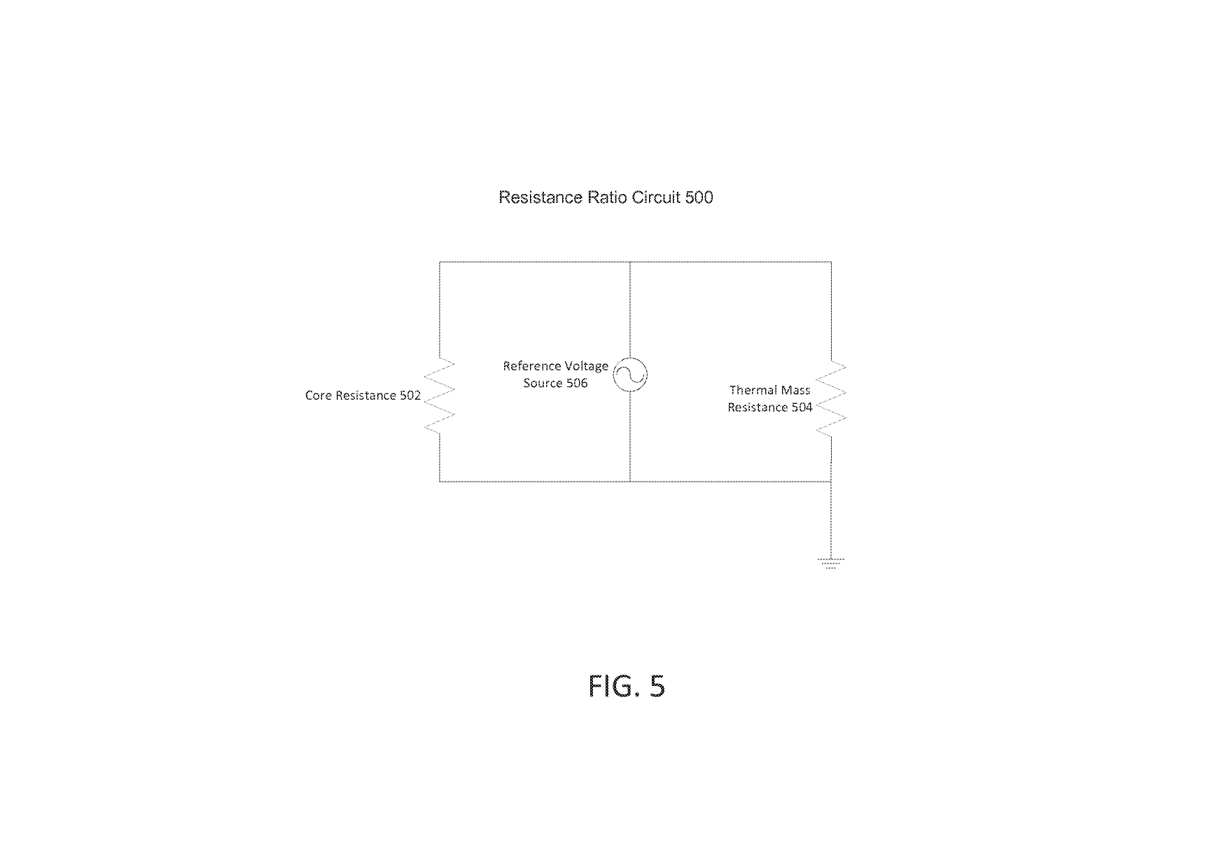

FIG. 5 is a schematic diagram of an electrical circuit 500 that is useful for modeling the behavior of core temperature sensor 300 of FIGS. 3A-C. Specifically, the resistance ratio circuit 500 can be used to determine the ratio of the thermal resistances of the tissue 326 and the thermal mass 308, which are depicted in FIG. 3A. The core temperature circuit 500 models temperatures as voltages, thermal resistances as electrical resistances, and thermal currents as electrical currents. The tissue 326 surrounding the organ 302 conducts and dissipates heat, and is therefore modeled as core resistance 502. Similarly, the thermal mass 308 is represented by thermal mass resistance 504. Finally, the heating element 316 combined with the ambient temperature corresponds to reference voltage 506.

When a voltage is applied by reference voltage source 506, current will travel through core resistance 502 and through thermal mass resistance 504. The ratio of current travelling through each resistive element in circuit 500 will be inversely proportional to the resistance of the element. For example, if the resistances of core resistance 502 and thermal mass resistance 504 are equal, then the current travelling through each element will be equal. However, if the thermal mass resistance 504 is larger than the core resistance 502, then more current will flow through core resistance 502 than through thermal mass resistance 504.

This same principle will can be applied to the core temperature sensor 300 of FIGS. 3A-C. The heating element 316 is located on the bottom surface of the body-contacting substrate 304, between the tissue 326 and the thermal mass 308. When a reference heat is applied by the heating element 316, some of the heat will be dissipated through the thermal mass 308, and the remaining heat will be dissipated through the tissue 326. As in the resistance ratio circuit 500, the amount of heat dissipated through the thermal mass 308 and the tissue 326 will be proportional to their thermal resistances. The amount of heat dissipated through the thermal mass 308 can be calculated by the differential temperature sensor 312, shown in FIGS. 3B-C. It can then be inferred that the remaining heat was dissipated through the tissue 326. The ratio of the thermal resistance of thermal mass 308 to the thermal mass of tissue 326 can be calculated as the inverse of the ratio of heat traveling through these elements.

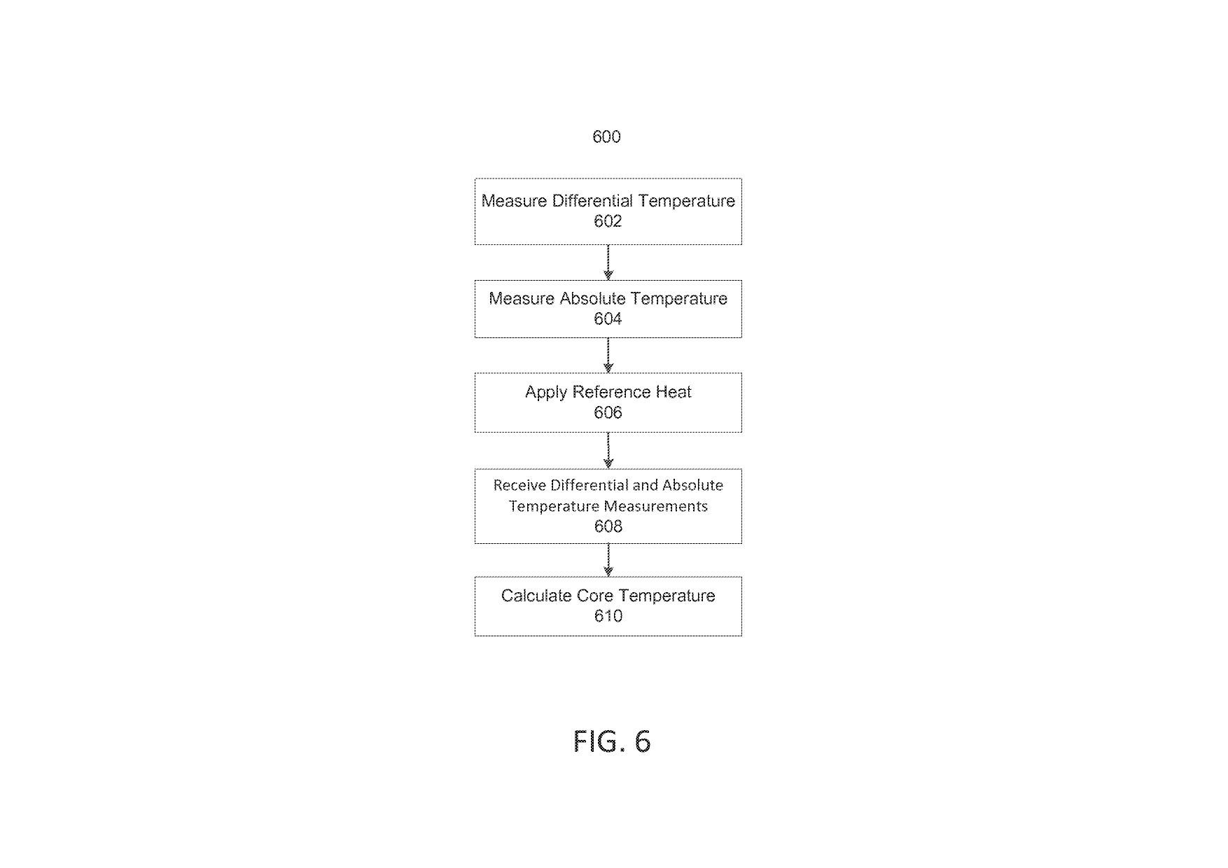

FIG. 6 is a flow diagram depicting a method 600 of determining a core temperature of an internal organ in a body. The method 600 includes the step of measuring, by a differential temperature sensor, a temperature difference across a thermal mass of known thermal resistance surrounded by a substrate (step 602). As depicted in FIGS. 2A-C and FIGS. 3A-C, the substrate has a body-contacting portion and an external portion, and the thermal mass is located between these two portions of the substrate.

The method 600 also includes the step of measuring, by an absolute temperature sensor, an absolute temperature at the external portion of the substrate (step 604). The external portion of the substrate has one surface in contact with the thermal mass and a second surface that is exposed to the surrounding environment, as shown in FIGS. 2A-C and FIGS. 3A-C. In one implementation, the external temperature sensor is a thermistor embedded into the external substrate.

The method 600 can also include the step of applying a reference heat to the substrate (step 606). The reference heat is applied by a heating element. As shown in FIGS. 2B-C and FIGS. 3B-C, the heating element can be located on either the body-contacting portion of the substrate or the external portion of the substrate. The reference heat applied by the heating element is a known heat applied uniformly to one portion of the substrate material. An electronic processor can select the reference heat to be applied and control the heating element to apply the selected heat. A power supply provides power to the heating element. In some implementations, the reference heat is a constant temperature. In other implementations, the reference heat changes over time (e.g., according to a modulation scheme).

The method 600 also includes the step of receiving, by an electronic processor, information corresponding to the temperatures measured by the differential and absolute temperature sensors (step 608). The electronic processor is logic circuitry that fetches and processes instructions stored within the processor. The processor is in communication with the differential temperature sensor and with the absolute temperature sensor, and the measurements from the temperature sensors are transmitted to the processor.

The method 600 also includes the step of calculating, by the electronic processor, a core temperature of the internal organ based on the temperatures measured by the absolute and differential temperature sensors, and the thermal resistances of the thermal mass and the tissue surrounding the organ (step 610). The calculation of the core temperature can be based on the equations described above in connection with FIGS. 4-5. For example, the processor can determine the heat flux through the thermal mass based on the temperature difference across the thermal mass, as measured by the differential temperature sensor. Using the thermal resistance of the thermal mass and the thermal resistance of the tissue surrounding the organ whose temperature is to be measured, the processor can determine a heat flux through the tissue. Finally, the processor can determine the core temperature of the organ based on the calculated heat flux through the tissue and the temperatures measured by the differential and absolute temperature sensors. In some implementations, the processor can calculate the relative thermal resistances of the thermal mass and the tissue surrounding the organ based on the measured response to application of the reference heat. The thermal resistance of the thermal mass can also be preselected to substantially match the thermal resistance of the tissue. In other implementations, the thermal resistance of the thermal mass is variable and controlled by the electronic processor. For example, the core temperature sensor can include a microfluidic pump and a reservoir containing liquid whose thermal resistance is different from the thermal resistance of the thermal mass, as described above. The electronic processor can control the microfluidic pump to allow a desired amount of liquid to be transported into the thermal mass, thereby controlling the overall thermal resistance of the thermal mass.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. An apparatus for measuring a core temperature of an internal organ in a body, the apparatus comprising:
a substrate having a body-contacting portion in contact with a surface of the body above the internal organ and an external portion opposed to the body-contacting portion and not in contact with the body;
a thermal mass of known thermal resistance, located between and in contact with the body-contacting portion and the external portion of the substrate;
a differential temperature sensor measuring a temperature difference between the body-contacting and external portions of the substrate;
an absolute temperature sensor measuring an absolute temperature at one of the body-contacting surface and the external portion of the substrate; and
an electronic processor configured to:
i) receive information corresponding to the temperatures measured by the differential and absolute temperature sensors; and
ii) calculate the core temperature of the internal organ based on the temperatures measured by the differential and absolute temperature sensors and the thermal resistances of the thermal mass and tissue surrounding the internal organ.

2. The apparatus of claim 1, further comprising an adhesive deposited on the body-contacting surface of the substrate for removably attaching the body-contacting substrate to the surface of the body.

3. The apparatus of claim 1, wherein the body-contacting and external portions of the substrate are connected in a continuous, U-shaped piece.

4. The apparatus of claim 1, wherein the body-contacting and external portions of the substrate are separate pieces, each attached to opposing ends of the thermal mass.

5. The apparatus of claim 1, further comprising an insulating element surrounding an exposed surface of the thermal mass.

6. The apparatus of claim 1, wherein the differential temperature sensor comprises a thermopile.

7. The apparatus of claim 1, wherein the electronic processor is further configured to determine the thermal resistance of the tissue surrounding the organ.

8. The apparatus of claim 1, further comprising a heating element for applying a reference heat to the body-contacting portion of the substrate.

9. The apparatus of claim 1, further comprising a heating element for applying a reference heat to the external portion of the substrate.

10. The apparatus of either claim 8 or claim 9, wherein the heating element comprises a carbon trace resistive heater.

11. The apparatus of claim 1, wherein the thermal mass has a thermal resistance substantially the same as the thermal resistance of the tissue surrounding the internal organ.

12. The apparatus of claim 1, wherein the electronic processor is further configured to control the thermal resistance of the thermal mass.

13. The apparatus of claim 1, wherein the thermal mass is composed of a plastic material.

14. A method for determining a core temperature of an internal organ in a body, the method comprising:
measuring, by a differential temperature sensor, a temperature difference across a thermal mass of known thermal resistance surrounded by a substrate having a body-contacting portion in contact with a surface of the body above the internal organ and an external portion opposed to the body-contacting portion and not in contact with the body;
measuring, by an absolute temperature sensor, an absolute temperature at the external portion of the substrate;
receiving, by an electronic processor, information corresponding to the temperatures measured by the differential and absolute temperature sensors; and
calculating, by the electronic processor, a core temperature of the internal organ based on the temperatures measured by the differential and absolute temperature sensors and the thermal resistances of the thermal mass and tissue surrounding the organ.

15. The method of claim 14, further comprising the step of applying, by a heating element, a reference heat to the body-contacting portion of the substrate.

16. The method of claim 14, further comprising the step of applying, by a heating element, a reference heat to the external portion of the substrate.

17. The method of claim 14, further comprising selecting the thermal resistance of the thermal mass to substantially match the thermal resistance of the tissue surrounding the internal organ.
